(19) United States
(12) Patent Application Publication
(10) Pub. No.: US 2004/0098043 A1
(43) Pub. Date: May 20, 2004

(54) Delivery apparatus for use during a surgical procedure and method of using the same

(76) Inventor: Hugh H. Trout III, Bethesda, MD (US)

Correspondence Address: COLLIER SHANNON SCOTT, PLLC, 3050 K STREET, NW, SUITE 400, WASHINGTON, DC, 20007, US

(21) Appl. No.: 10/614,782

(22) Filed: Jul. 9, 2003

Related U.S. Application Data
(60) Provisional application No. 60/394,233, filed on Jul. 9, 2002.

(51) Int. Cl. A61B 17/08

(52) U.S. Cl. 606/213

(57) ABSTRACT

The present invention is directed to a delivery apparatus for performing a surgical procedure. The delivery apparatus comprises: a flexible catheter, a penetration apparatus; and at least one fastener. The flexible catheter is capable of assuming an angular configuration at a predetermined time during the surgical procedure. The penetration apparatus is disposed within the catheter and further comprises a first end having a tip and a second end that is substantially free. The fastener is housed in the penetration apparatus. The apparatus further comprises a sealant material associated with at least one potion of the fastener.

### CROSS-REFERENCE TO RELATED APPLICATION

The present invention relates to, and is entitled to the benefit of the earlier filing date and priority of, Application No. 60/394,233, filed on Jul. 9, 2002, which is herein incorporated by reference as if fully set forth.

### FIELD OF THE INVENTION

The present invention relates to a delivery apparatus. In particular, the present invention is directed to a delivery apparatus for use in a surgical procedure wherein at least one fastener is delivered to a site of the surgical procedure by the delivery apparatus.

### BACKGROUND

An aneurysm is a ballooning of the wall of an artery resulting from the weakening of the artery due to disease or other conditions. Left untreated, an aneurysm may rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of such surgery. It is, therefore, desirable to reduce the mortality and morbidity associated with surgical intervention.

In recent years, methods have been developed to attempt to treat an aortic aneurysm without the attendant risks of intra-abdominal surgical intervention. Among them are inventions disclosed and claimed in Kornberg, U.S. Pat. No. 4,562,596 for Aortic Graft, Device and Method for Performing an Intraluminal Abdominal Aortic Aneurysm Repair; Lazarus, U.S. Pat. No. 4,787,899 for Intraluminal Graft Device, System and Method; and Taheri, U.S. Pat. No. 5,042,707 for Intravascular Stapler, and Method of Operating Same.

Although in recent years certain techniques have been developed that may reduce the stress, morbidity, and risk of mortality associated with surgical intervention to repair aortic aneurysms, none of the systems that have been developed provide methods and apparatus directed to the repair of thoracic aneurysms. Treatment of patients with thoracic, thoracoabdominal or upper abdominal aortic aneurysms is difficult. The open operative procedures have a high mortality rate with an associated serious and frequent morbidity as well. Accordingly, the endovascular approach holds much promise as an alternative treatment method. Use of fasteners that penetrate the prosthetic graft as well as the full thickness of the aortic wall will undoubtedly facilitate an endovascular approach. Such an approach carries with it the possibility of causing bleeding through the holes in the aorta; moreover, any penetrating method has the risk of puncturing a lung and causing a pneumothorax. While a small air leak from a traumatized lung would necessitate insertion of a tube in the chest, this minor procedure would be far less traumatic than an open aneurysm repair. Nonetheless, minimizing lung trauma as well as minimizing blood loss into the chest, are desirable attributes of an endovascular approach to treatment of patients with thoracic, thoracoabdominal or upper abdominal aortic aneurysms.

It is therefore an advantage of some, but not necessarily all, embodiments of the present invention to provide methods and apparatus for the surgical repair of aneurysms. It is another advantage of embodiments of the present invention to provide methods and apparatus for the surgical repair of thoracic, thoracoabdominal, and upper abdominal aneurysms.

Additional advantages of various embodiments of the invention are set forth, in part, in the description that follows and, in part, will be apparent to one of ordinary skill in the art from the description and/or from the practice of the invention.

### SUMMARY OF THE INVENTION

Responsive to the foregoing challenges, Applicant has developed an innovative delivery apparatus for use in a surgical procedure. According to at least one embodiment of the present invention, a delivery apparatus for performing a surgical component comprises: a flexible catheter capable of assuming an angular configuration at a predetermined time during the surgical procedure; a penetration apparatus disposed within the catheter, the penetration apparatus further comprises a first end having a tip, and a second end that is substantially free; and at least one fastener in communication with the penetration apparatus.

According to at least another embodiment of the present invention, the delivery apparatus for performing a surgical component comprises: a flexible catheter capable of assuming an angular configuration at a predetermined time during the surgical procedure; a penetration apparatus disposed within the catheter, the penetration apparatus further comprises a first end having a tip, and a second end that is substantially free; at least one fastener in communication with the penetration apparatus; and a sealant material associated with at least a portion of the fastener.

According to at least one further embodiment of the present invention, the delivery apparatus for performing a surgical component comprises: a flexible catheter capable of assuming an angular configuration at a predetermined time during the surgical procedure; a penetration apparatus disposed within the catheter, the penetration apparatus further comprises a first end with a tip that is an open core needle and a second end that is substantially free; at least one fastener disposed within the penetration apparatus; and a sealant material associated with at least a portion of the fastener.

According to at least one additional embodiment of the present invention, the method for performing a surgical procedure at a surgical site, which comprises the steps of: advancing a delivery apparatus to the surgical site; activating the delivery apparatus to contact the surgical site; advancing the delivery apparatus wherein the delivery apparatus creates an aperture at the surgical site; and releasing a fastener to the surgical site, wherein at least one portion of the fastener further comprises a sealant material.

It is to be understood that both the foregoing general description and the following detailed description are exemplary and exemplary only, and are not restrictive of the invention as claimed.

### BRIEF DESCRIPTION OF THE DRAWINGS

In order to assist the understanding of this invention, reference will now be made to the appended drawings, in which like reference characters refer to like elements.

FIG. 1 is a cut-away pictorial view of an embodiment of the present invention at a surgical site.

FIGS. 2 and 3 are cut-away pictorial views of embodiments of the present invention advancing towards the surgical site.

FIG. 4 is a cut-away pictorial view of an embodiment of the tip penetrating through a surgical component and a vessel.

FIG. 5 is a cut-away pictorial view of the advancement of a fastener through a surgical component and a vessel.

FIG. 6 is a cut-away pictorial view of the deployment of a fastener of the present invention.

FIG. 7 is a cut-away pictorial view of an embodiment of the flexible catheter retracted from the inner catheter.

FIG. 8 is a cut-away pictorial view of the retraction of the delivery apparatus with the fastener deployed at the surgical site.

FIG. 9 is a cut-away pictorial view of an example embodiment of the delivery apparatus.

FIGS. 10 and 11 are cut-away pictorial views of example embodiments of the present invention advancing towards the surgical site.

FIG. 12 is a cut-away pictorial view of an embodiment of the tip penetrating through a surgical component and a vessel.

FIG. 13 is a cut-away pictorial view of the advancement of a fastener through a surgical component and a vessel.

FIG. 14 is a cut-away pictorial view of the deployment of a fastener of the present invention.

FIG. 15 is a cut-away pictorial view of the retraction of the delivery apparatus with the fastener deployed at the surgical site.

FIG. 16 is a cut-away pictorial view of example embodiments of fasteners used in the delivery apparatus.

FIG. 17 is a cut-away pictorial view of an embodiment of a fastener deployed at a surgical site.

FIG. 18 is a cut-away pictorial view of a further embodiment of the present invention penetrated through a surgical component and a vessel.

FIGS. 19, 20, and 21 are cut-away pictorial views of the present invention deploying fasteners.

FIGS. 22 and 23 are cut-away pictorial views of the retraction of the delivery apparatus after deploying an fastener to a surgical site.

### DETAILED DESCRIPTION OF EMBODIMENTS OF THE INVENTION

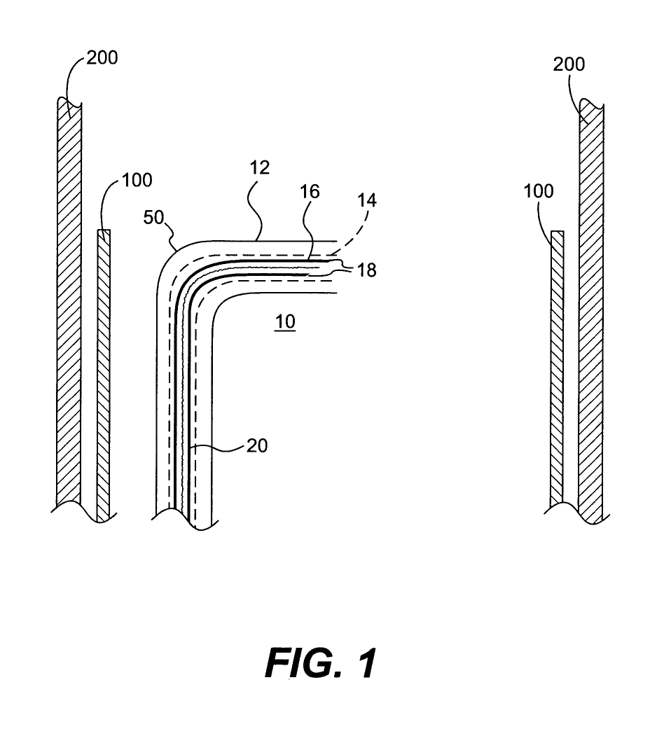

Reference will now be made in detail to embodiments of the present invention, examples of which are illustrated in the accompanying drawings. With reference to FIGS. 1-8, the delivery apparatus 10 is shown in a pictorial cross-section. The delivery apparatus 10 may comprise a flexible catheter 12, a penetration apparatus 16, a tip 18 of the penetration apparatus 16, and at least one or more fasteners 20. The penetration apparatus 16 may have a hollow core or a solid core. In some embodiments, the delivery apparatus 10 may also comprise an inner catheter 14 within the flexible catheter 12, as shown in FIG. 1.

The delivery apparatus 10 advances through a vessel (not shown) to a surgical site within the vessel to insert at least one or more fasteners 20 through a surgical component 100 and a vessel 200. The advancement of the delivery apparatus 10 is through, but not limited to, the extension of the delivery apparatus 10 by a mechanical mechanism, by hand, or by any other suitable means.

In an example embodiment of FIG. 1, the flexible catheter 12 has an end portion 50 capable of assuming an angular configuration. In at least one embodiment, the end portion 50 may be manipulated by using a pull wire (not shown), as disclosed in U.S. patent application Ser. No. 09/783,313 filed on Feb. 15, 2001, which is herein incorporated in its entirety by reference. The delivery apparatus 10, as a whole, is flexible such that it may be multi-directional to enable maneuvering through tortuous vessels. In further example embodiments, the flexible catheter 12 articulates wherein an end portion of the flexible catheter 12 assumes an angular configuration, as illustrated in FIG. 1. In other example embodiments, an expandable member such as, but not limited to, a balloon, or additional articulation of the flexible catheter 12 to create an appositional force, or any other suitable means may be used in conjunction with the flexible catheter 12 (not shown). In the embodiment of FIG. 1, a surgical component 100 is attached to a vessel 200. In alternative embodiments, the surgical component may be attached to another surgical component, or tissue attached to tissue, or attachment of any combination of surgical components, vessels, and tissues.

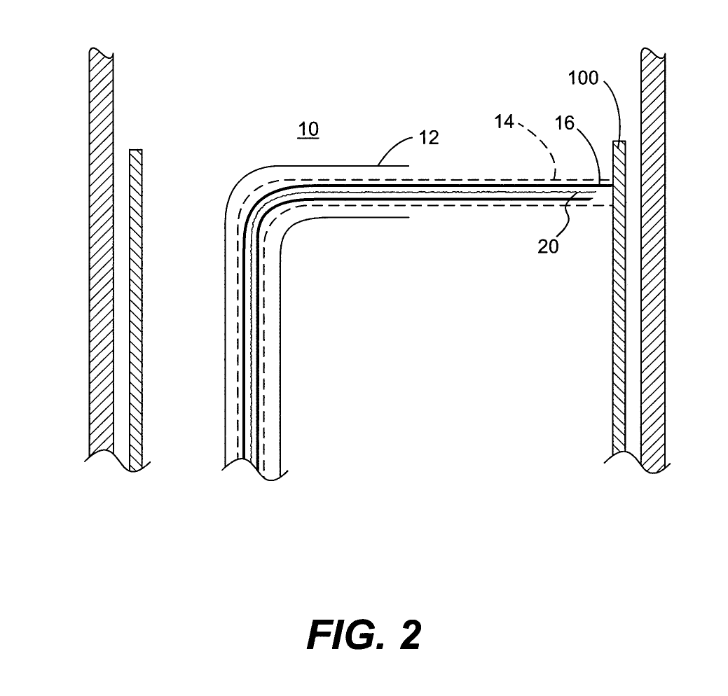

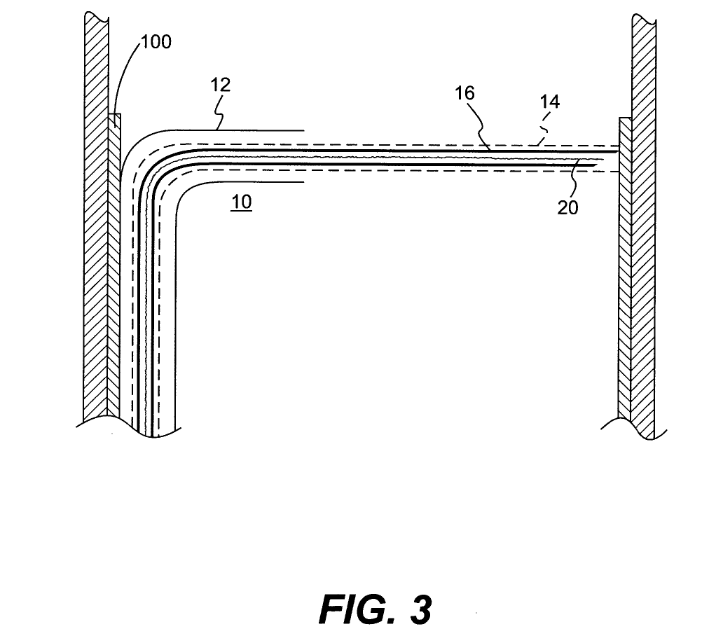

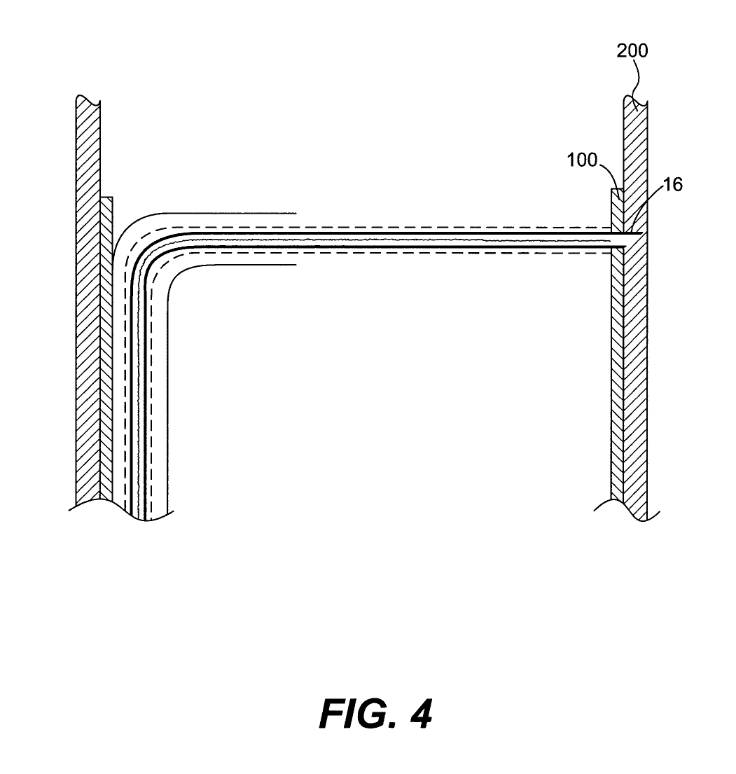

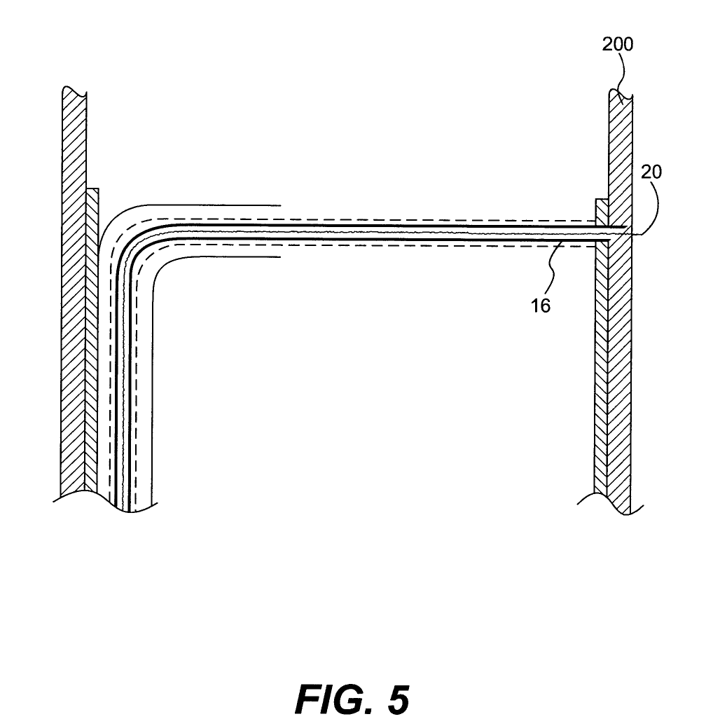

An embodiment of the method of the delivery apparatus 10 will now be described with reference to FIGS. 2-8. In FIG. 2, the delivery apparatus 10 is advanced to the surgical site through the vessel (not shown). The inner catheter 14 along with the penetration apparatus 16 are advanced either simultaneously or in sequence such that they extend from the flexible catheter 12. The inner catheter 14 and/or the penetration apparatus 16 may be advanced further until applying sufficient pressure on the surgical component 100 to push the surgical component 100 against the vessel 200, as shown in FIG. 3. In further example embodiments, with the advancement of the inner catheter 14 and/or the penetration apparatus 16, the flexible catheter 12 abuts the opposite side of the surgical component 100, also depicted in FIG. 3. The tip 18 of the penetration apparatus 16 is then activated and advanced to create an aperture in the surgical component 100 and the vessel 200, illustrated in FIG. 4. The penetration apparatus 16 and the tip 18 advance to penetrate the surgical component 100 and the vessel 200 to create an aperture there through. The activation of the tip 18 and the penetration apparatus 16 occurs by, but is not limited to, mechanical or electrical mechanisms or any other appropriate mechanism. Through the aperture, the fastener 20 extends spanning the surgical component 100 and the vessel 200, as shown in FIG. 5. One or more fasteners may be deployed from the hollow core of the penetration apparatus 16 or deployed from around an outside surface of the penetration apparatus 16.

In example embodiments of the present invention, the fastener 20 is associated with a sealant material such as, but not limited to, a plug of the surgical component, an absorbable substance, an occluding substance, or any other suitable material or any combination thereof. In further example embodiments, the absorbable substance may be, but not limited to, Gelfoam® or Surgicil®). The use of the sealant substance may be beneficial in preventing or minimizing bleeding when inserted into the surgical component and the vessel with or without the fastener.

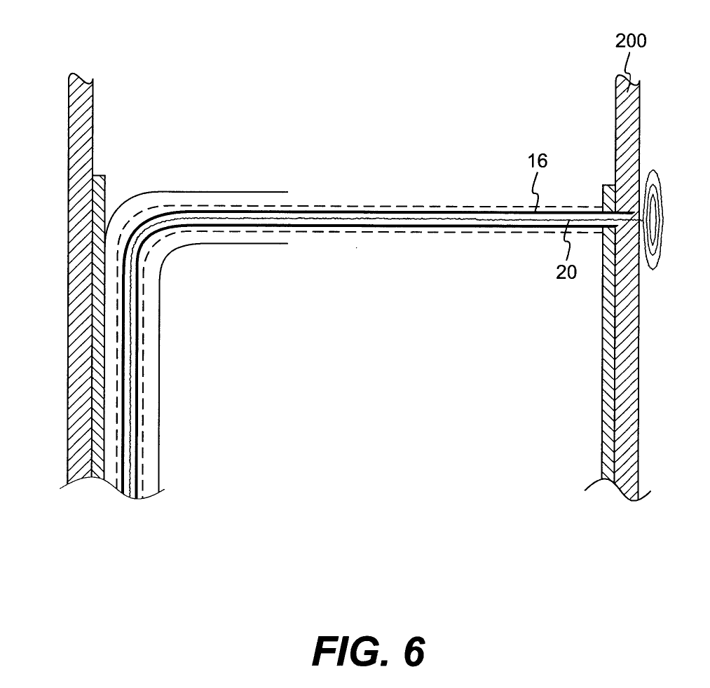

The fastener 20 in FIG. 6 is flexible applying a force to secure the surgical component 100 to the vessel 200, as disclosed in the following U.S. patents and patent applications: U.S. Provisional Patent Application No. 60/181,230, filed Feb. 9, 2000; U.S. patent application Ser. No. 09/442,768, filed Nov. 18, 1999; U.S. patent application Ser. No. 09/213,233, filed Dec. 17, 1998, now U.S. Pat. No. 5,997,556; U.S. patent application Ser. No. 08/958,524, filed Oct. 27, 1997, now U.S. Pat. No. 5,957,940; U.S. patent application Ser. No. 08/896,415, filed Jul. 18, 1997, now U.S. Pat. No. 5,944,750; and U.S. Provisional Patent Application No. 60/051,209, filed Jun. 30, 1997. The subject matter of the patents and patent applications are incorporated herein specifically by reference.

In various example embodiments, the tip 18 of the penetration apparatus 16 comprises an open core needle, a leading edge, a heating probe, a radio frequency tip, a boring tip, a coring tip, an optical fiber, or any other suitable means capable of penetrating through the surgical component and/or tissues or vessel. In FIG. 6, the tip 18 penetrating through the surgical component 100 and the vessel 200 is an open core needle tip. Because the open core needle tip is sharp and pointed, penetration occurs with the needle's advancement. In further example embodiments, the tip 18 is interchangeable such that the tip is integral with the penetration apparatus 16 and able to accommodate varying thicknesses and densities of the surgical site. In other example embodiments, the tip 18 may be an extension of the penetration apparatus 16 such that the tip 18 and the penetration apparatus 16 are a unitary structure and/or of unitary construction.

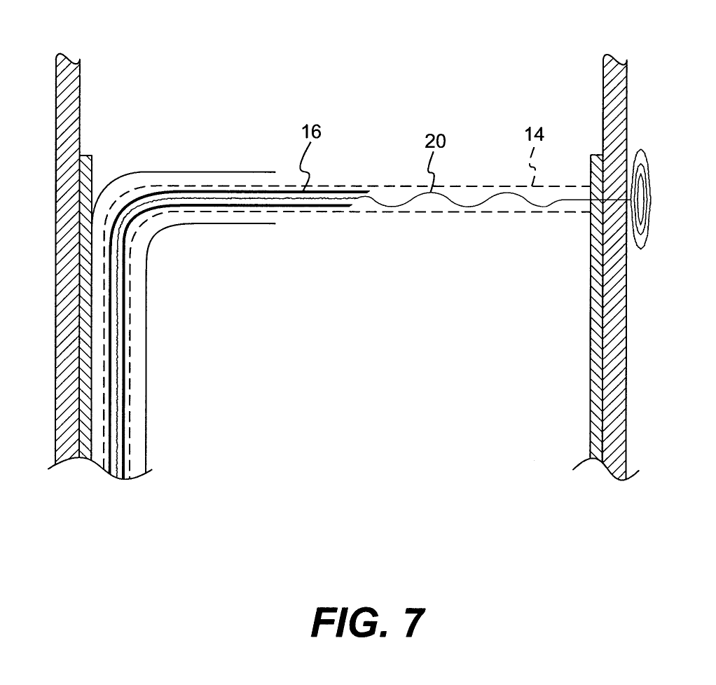

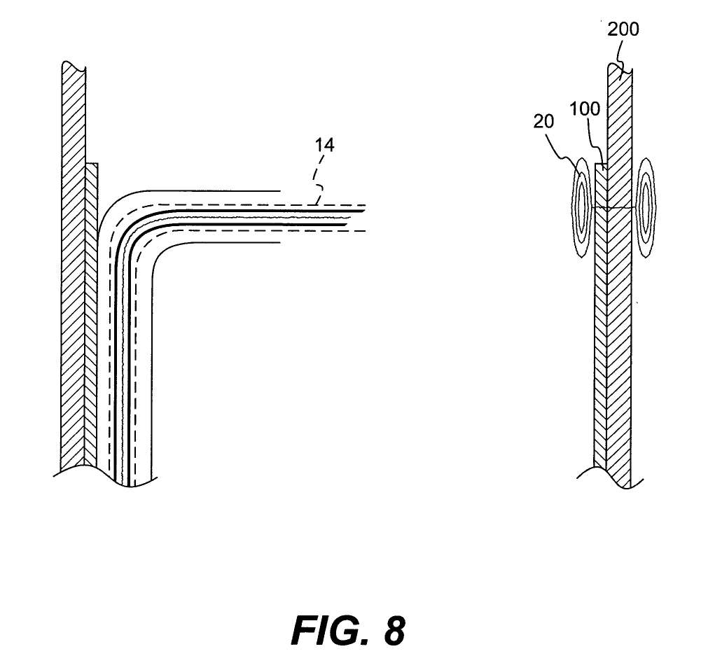

In FIG. 7, the fastener 20 within the inner catheter 14 starts to assume its unconstrained configuration but may be precluded from doing so completely by the inner catheter 14. With the withdrawal of the inner catheter 14, the fastener 20 assumes an unconstrained configuration. As shown in FIG. 8, the penetration apparatus 16 and the inner catheter 14 are retracted toward the flexible catheter 12, thereby deploying the fastener 20 to secure the surgical component 100 to the vessel 200. In some embodiments, this method may be repeated at various locations of the surgical site to deploy multiple fasteners.

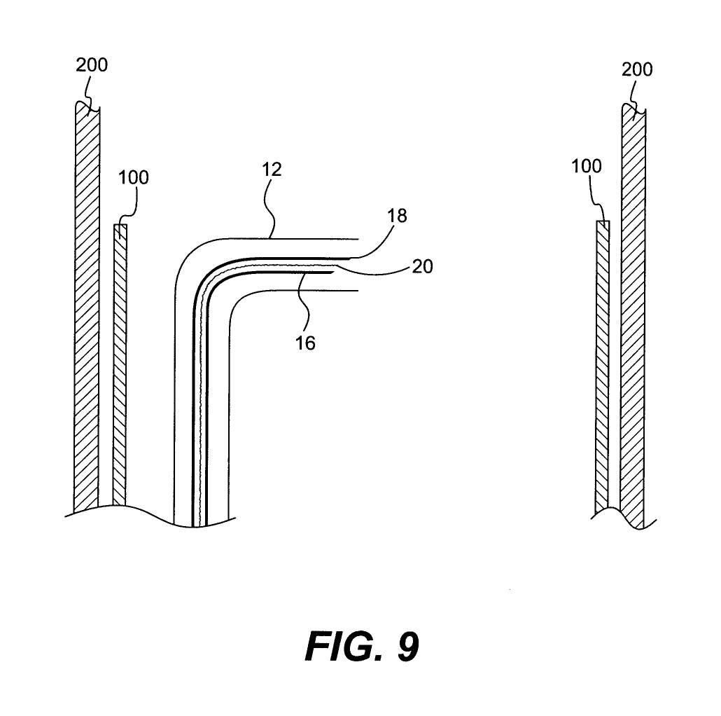

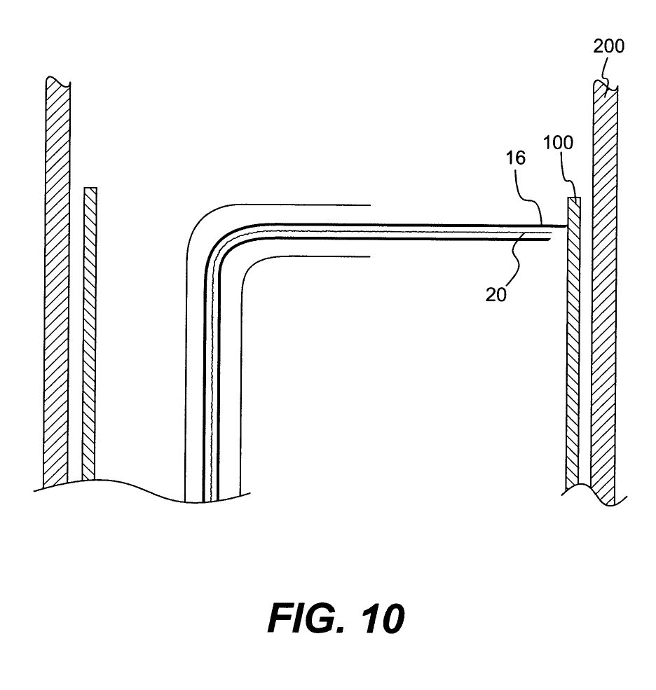

Further example embodiments of the present invention are illustrated in FIGS. 9-14. In FIGS. 9-14, the delivery apparatus 10 may be advanced through the lumen of a surgical component 100, such as, but not limited to, a prosthetic graft, which is positioned adjacent to the vessel 200. In FIG. 9, the delivery apparatus 10 comprises a flexible catheter 12, a penetration apparatus 16, a tip 18 of the penetration apparatus 16, and at least one fastener 20. The penetration apparatus 16 is advanced to the surgical site such that the tip 18 contacts the surgical component 100, as illustrated in FIG. 10.

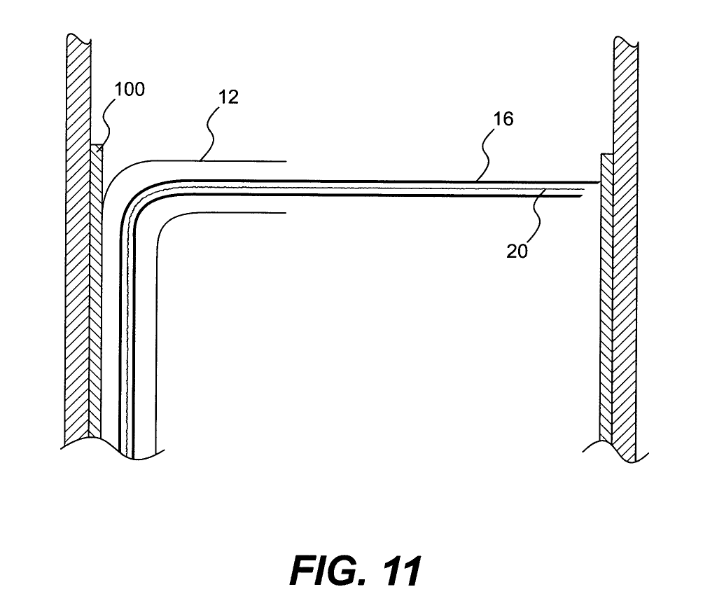

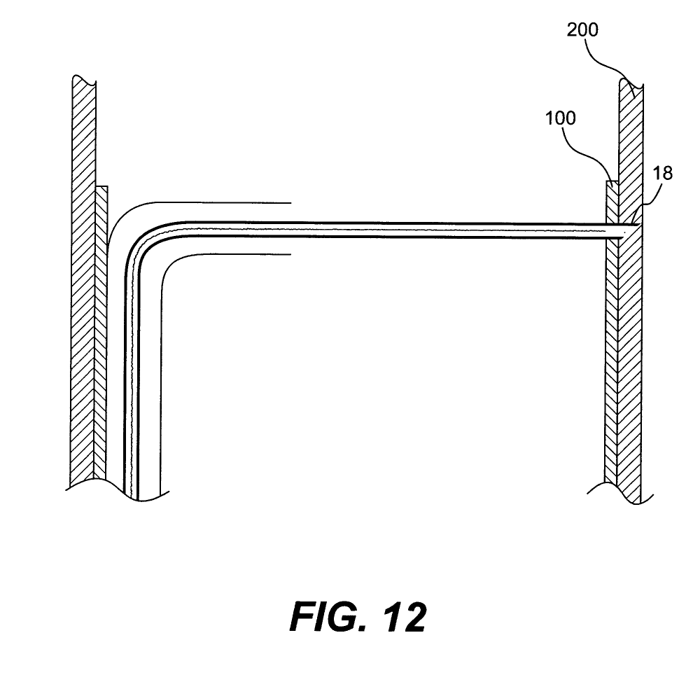

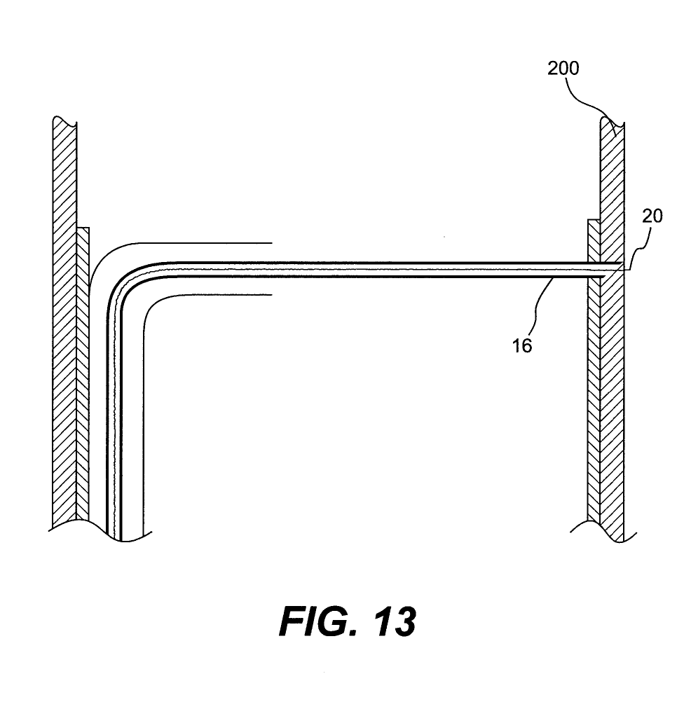

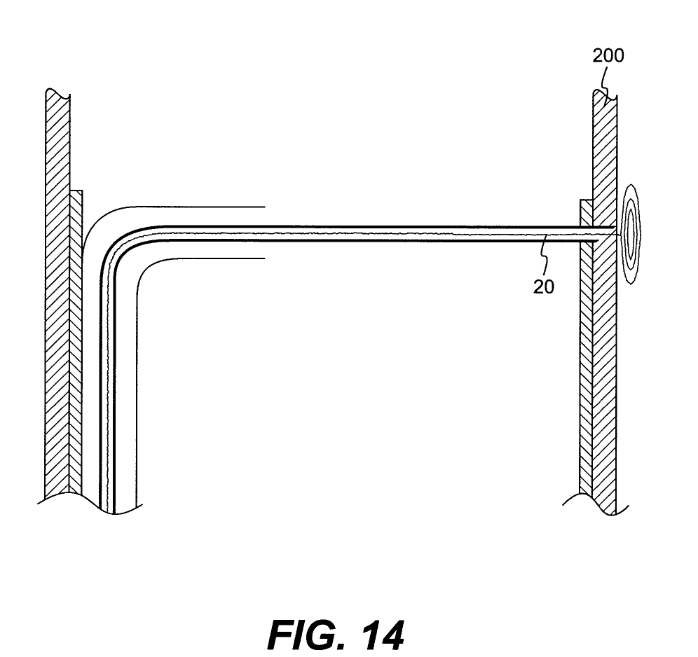

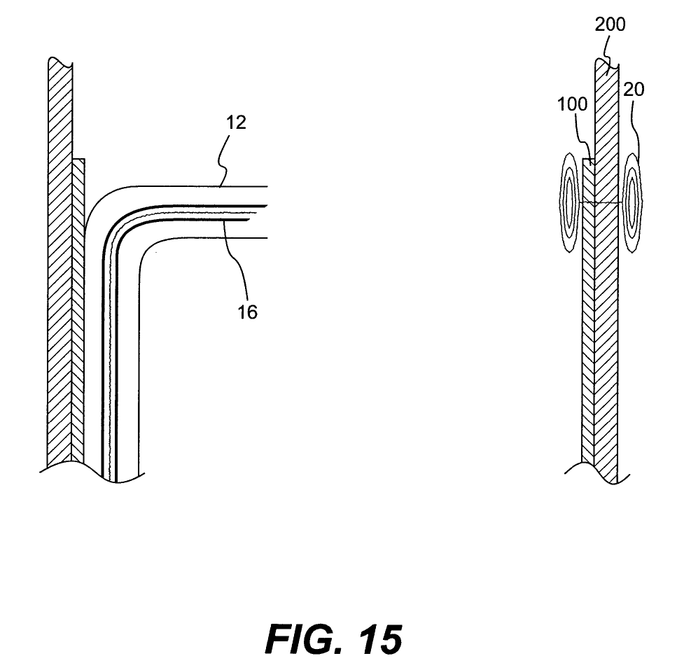

In FIG. 10, the penetration apparatus 16 is advanced such that it extends to contact the surgical component 100. The penetration apparatus 16 still advances to push the surgical component 100 against the vessel 200, as shown in FIG. 11. The penetration apparatus 16 continues such that the tip 18 penetrates the surgical component 100 and the vessel 200, as depicted in FIG. 12. Once the tip 18 has sufficiently penetrated the surgical component 100 and the vessel 200, the fastener 20 is activated, illustrated in FIG. 13. In FIG. 13, the activation of the fastener 20 sequentially dispenses the fastener or a plurality of fasteners at the surgical site. Upon retraction of the penetration apparatus 16, the fastener 20 assumes a secondary orientation, securely attaching the surgical component 100 to the vessel 200 as shown in FIG. 13. In alternative embodiments, the fastener 20 is positioned within, around, or in conjunction with the penetration apparatus 16. In FIG. 14, the fastener 20 is illustrated in an unconstrained configuration outside the vessel 200. The fully released fastener spans the surgical component 100 and the vessel 200, as shown in FIG. 15.

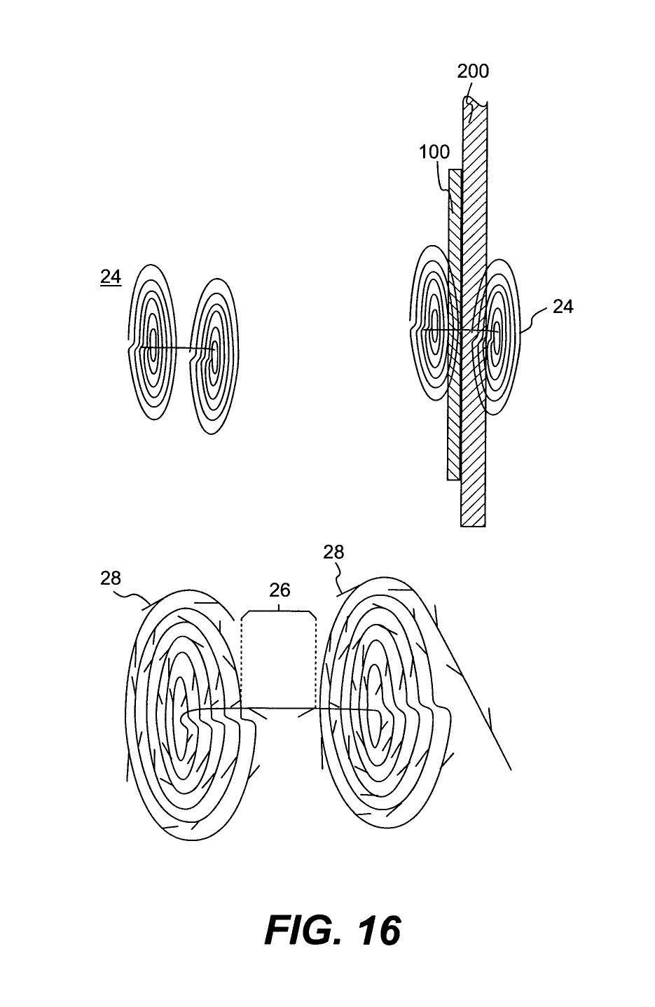

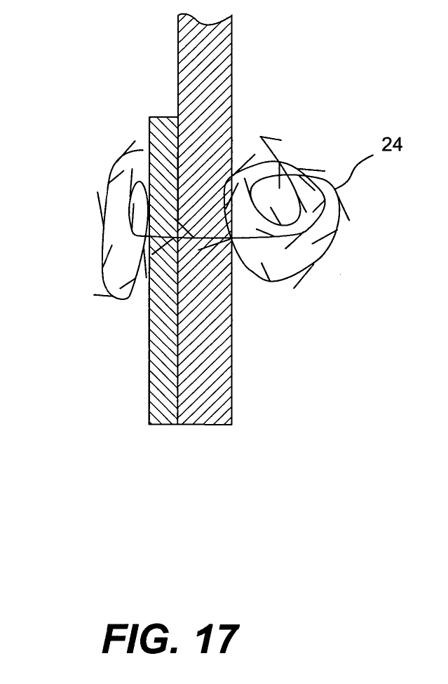

In various example embodiments illustrated in FIG. 16, the fastener 20 may be a double coil fastener 24 with minimal separation 26 between the coils. In some embodiments, the separation 26 may be, but is not limited to, about 0.5 mm to about 0.2 mm. The fastener 20 may also have barbs 28 located along the length of the fastener. The barbs 28 may be oriented so that the barb located on a portion of the coil outside the surgical component 100 and the vessel 200 will likely snag on an outside surface of the surgical component 100 and/or the vessel 200, as shown in FIG. 17.

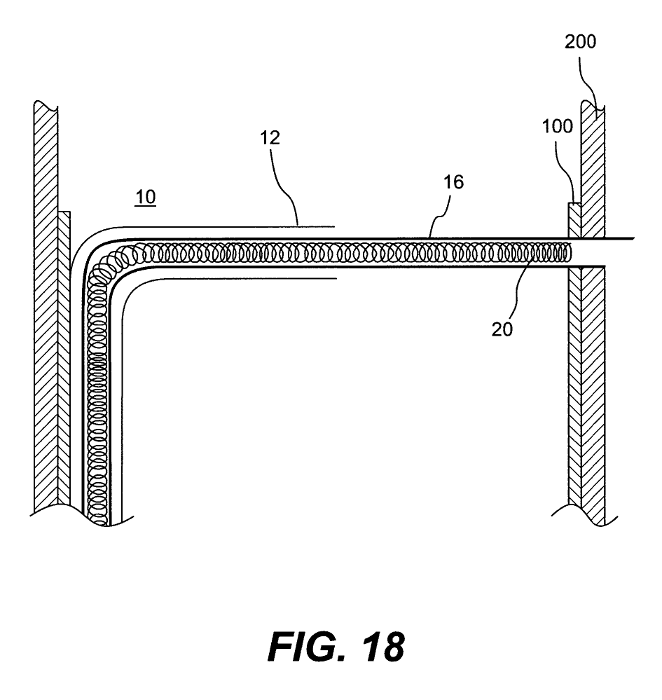

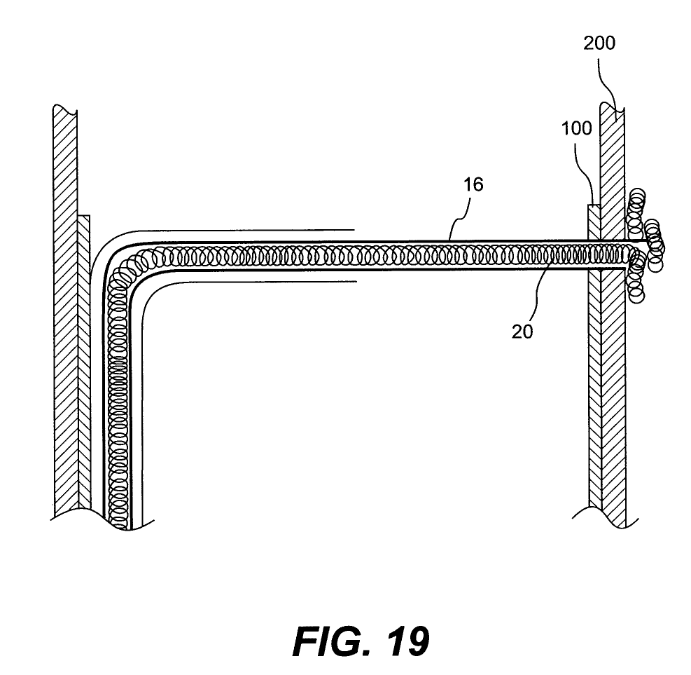

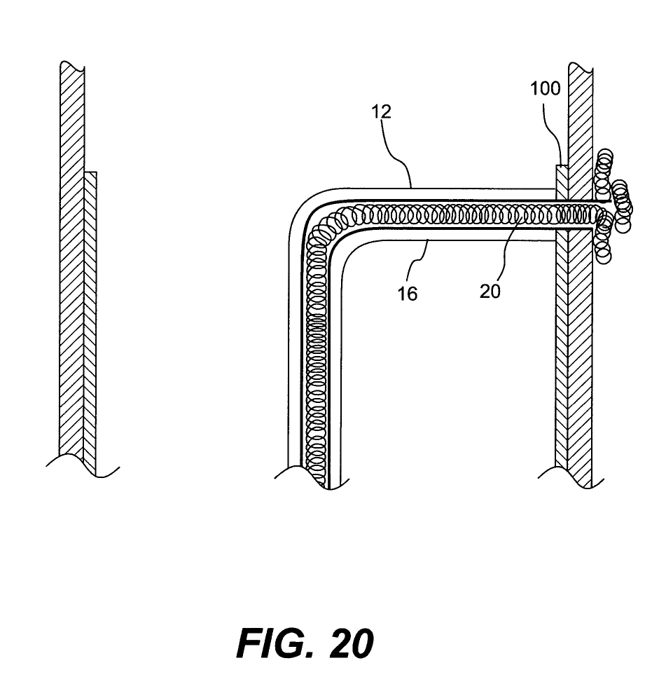

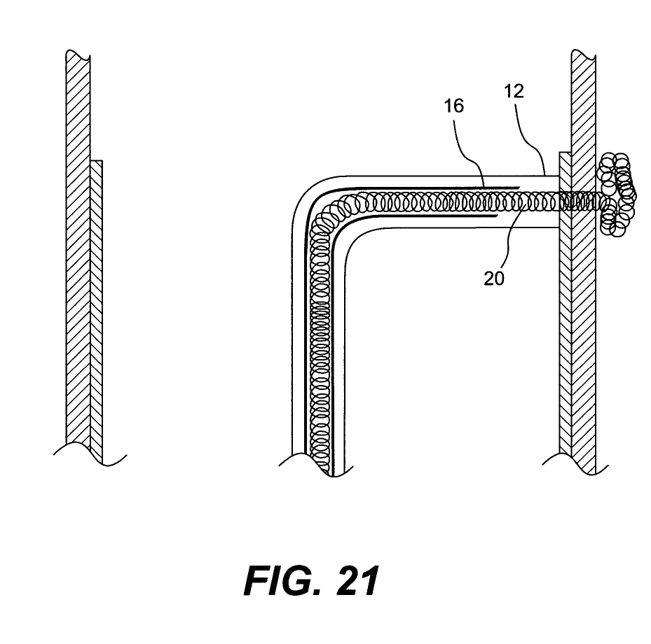

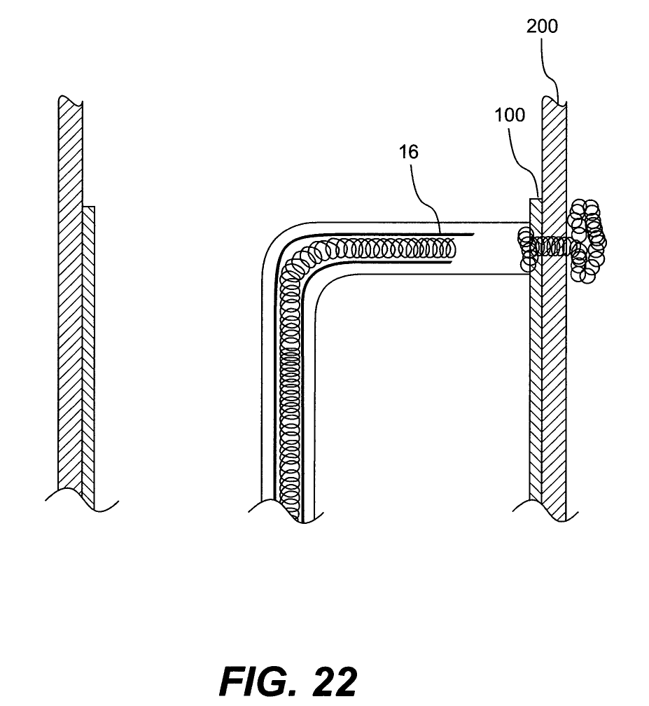

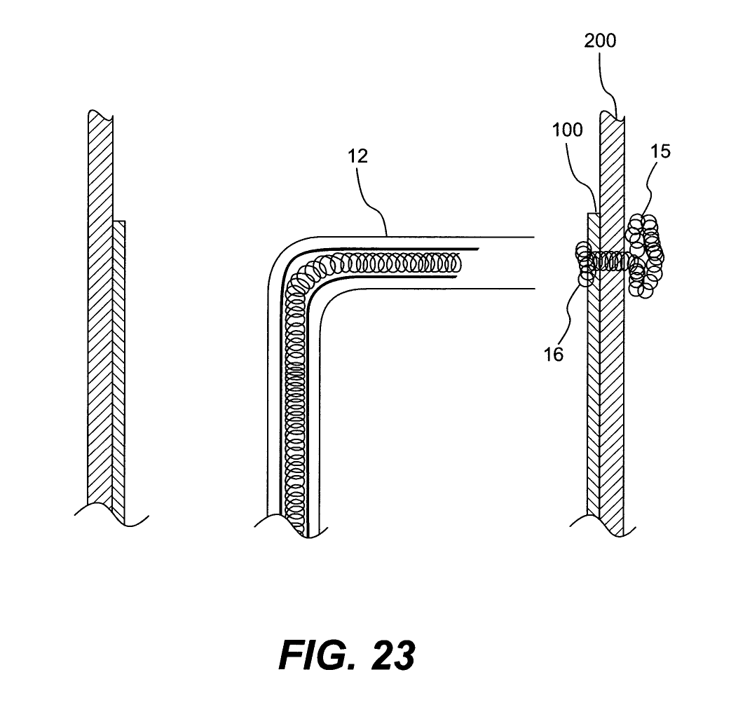

In FIGS. 18-23, an embodiment of the present invention depicts the delivery apparatus 10 with a coiled coil fastener, the fastener 20, being inserted through the surgical component 100 and the vessel 200. The insertion is by the tip 18 of the penetration apparatus 16. As the coil emerges from the penetration apparatus 16, it may assume its unconstrained coiled coil shape as seen in FIG. 19. In the embodiment shown in FIGS. 18-23, the flexible catheter 12 is retracted back to the penetration apparatus 16 until the flexible catheter 12 contacts the penetrated surgical component 100. As the penetration apparatus 16 is withdrawn back into the flexible catheter 12, the fastener 20 extrudes from the penetration apparatus 16, as seen in FIG. 21. As the penetration apparatus is withdrawn further, the coiled coil fastener 20 releases from the sheath 16 to assume its coiled coil configuration, depicted in FIG. 22. The flexible catheter 12 is then dislodged from the surgical component 100 such that the fastener 20 transverses the surgical component 100 and the vessel 200 with a coiled coil on both the extraluminal portion of the vessel 200 and the intraluminal portion of the surgical component 100.

Numerous characteristics and advantages have been set forth in the foregoing description, together with details of structure and function. The novel features are pointed out in the appended claims. The disclosure, however, is illustrative only, and changes may be made in detail, especially in matters of shape, size and arrangement of parts, within the principle of the invention, to the full extent indicated by the broad general meaning of the appended claims that are expressed. Further, it will be apparent to those skilled in the art that variations and modifications of the present invention can be made without departing from the scope or spirit of the invention.

What is claimed is:

1. A delivery apparatus for performing a surgical procedure comprising:
a flexible catheter capable of assuming an angular configuration at a predetermined time during the surgical procedure;
a penetration apparatus disposed within the catheter, the penetration apparatus further comprises a first end having a tip for creating an aperture, and a second end that is substantially free; and
at least one fastener in communication with the penetration apparatus.

2. The apparatus of claim 1, wherein the flexible catheter has an outer catheter and an inner catheter.

3. The apparatus of claim 1, wherein the tip of the penetration apparatus is a hollow core needle.

4. The apparatus of claim 1, further comprising a sealant material associated with at least a portion of the fastener.

5. The assembly of claim 4, wherein the sealant material is an occluding substance.

6. The assembly of claim 4, wherein the sealant material is a plug.

7. The assembly of claim 4, wherein the sealant material is an absorbent material.

8. A delivery apparatus for performing a surgical procedure comprising:
a flexible catheter capable of assuming an angular configuration at a predetermined time during the surgical procedure;
a penetration apparatus disposed within the catheter, the penetration apparatus further comprises a first end having a tip for creating an aperture, and a second end that is substantially free;
at least one fastener in communication with the penetration apparatus; and
a sealant material associated with at least a portion of the fastener.

9. The apparatus of claim 8, wherein the flexible catheter has an outer catheter and an inner catheter.

10. The assembly of claim 8, wherein the sealant material is an occluding substance.

11. The assembly of claim 8, wherein the sealant material is a plug.

12. The assembly of claim 8, wherein the sealant material is an absorbent material.

13. A delivery apparatus for performing a surgical procedure comprising:
a flexible catheter capable of assuming an angular configuration at a predetermined time during the surgical procedure;
a penetration apparatus disposed within the catheter, the penetration apparatus further comprises a first end with a tip for creating an aperture that is an open core needle and a second end that is substantially free;
at least one fastener disposed within the penetration apparatus; and
a sealant material associated with at least a portion of the fastener.

14. The apparatus of claim 13, wherein the flexible catheter has an outer catheter and an inner catheter.

15. The assembly of claim 13, wherein the sealant material is an occluding substance.

16. The assembly of claim 13, wherein the sealant material is a plug.

17. The assembly of claim 13, wherein the sealant material is an absorbent material.

18. A method for performing a surgical procedure at a surgical site, which comprises the steps of:
advancing a delivery apparatus to the surgical site;
activating the delivery apparatus to contact the surgical site;
advancing the delivery apparatus wherein the delivery apparatus creates an aperture at the surgical site; and
releasing a fastener to the surgical site, wherein at least one portion of the fastener further comprises a sealant material.
